(19) United States
(12) Patent Application Publication
(10) Pub. No.: US 2021/0038874 A1
(43) Pub. Date: Feb. 11, 2021

(54) BLOOD APPLICATOR FOR TISSUE TREATMENT

(71) Applicant: REDDRESS LTD., Pardes Hana (IL)

(72) Inventors: Igal KUSHNIR, Pardes Hana (IL); Alon KUSHNIR, Givat Ada (IL)

(73) Assignee: REDDRESS LTD., Pardes Hana (IL)

(21) Appl. No.: 16/966,187

(22) PCT Filed: Nov. 26, 2018
(86) PCT No.: PCT/IL2018/051279

§ 371 Date: Jul. 30, 2020

(30) Foreign Application Priority Data
Jan. 30, 2018 (IL) 257229

(51) Int. Cl. A61M 35/00; A61B 5/15

(52) U.S. Cl. CPC A61M 35/003; A61M 11/007; A61M 2202/0413; A61B 5/150343

(57) ABSTRACT

An applicator for use in tissue treatment, a method and a kit using the same. The applicator includes (i) a first chamber for holding whole blood, (ii) a second chamber for holding a coagulation initiator, and (iii) a dispensing element configured for concomitantly receiving, upon actuating the applicator, a volume of whole blood from said first chamber and a volume of coagulation initiator from said second chamber and dispensing the two together onto a tissue in need of treatment.

### TECHNOLOGICAL FIELD

The present disclosure concerns tissue treatment and in particular treatment of tissue making use of blood, preferably, whole blood.

### BACKGROUND ART

References considered to be relevant as background to the presently disclosed subject matter are listed below:
U.S. Pat. No. 9,180,142
US Patent Application Publication No. 2017/0056450
U.S. Pat. No. 8,728,466

Acknowledgement of the above references herein is not to be inferred as meaning that these are in any way relevant to the patentability of the presently disclosed subject matter.

### BACKGROUND

The use of blood or blood components for treating wounds has been described.

U.S. Pat. No. 9,180,142 describes wound dressing prepared from whole blood that is clotted ex vivo to form a sheet of clotted blood that is applied onto the skin over a sight of skin injury.

EP Patent Application Publication No. 3136026 describes a combined autologous biologic and cold therapy treatment for treating various skin injuries. The treatment may include applying autologous blood components, including but not limited to platelet rich plasma, to a skin injury in a manner that influences the healing process. A tray assembly for freezing autologous blood components includes a tray body and a plurality of compartments formed in the tray body. Each of the plurality of compartments is configured to receive an individual dose of an autologous blood component.

U.S. Pat. No. 8,728,466 describes a method for treating a second or third degree skin burn of a mammal with reduced risk of hypertrophic scarring, wherein said method comprises applying a skin graft, platelet rich plasma, and a thrombin preparation to at least a portion of the burn site of said second or third degree skin burn, and wherein said portion of said burn site heals with no visible hypertrophic scarring.

### General Description

The present disclosure is based on the finding that tissue can be regenerated by application of whole blood directly on the tissue in need of regeneration, the whole blood preferably being combined, prior to application, with a coagulation initiator such that its application onto the tissue is concomitant with clotting of the whole blood.

Based, inter alia, on the above finding, three aspects are provided by this disclosure: one concerns an applicator for tissue treatment comprising at least whole blood; a second aspect concerns a method of tissue treatment comprising application of at least whole blood; and a third concerns a kit comprising the applicator for use in tissue treatment. All three aspects center around the direct application of whole blood onto a tissue in need of treatment.

Specifically, the present disclosure provides an applicator for tissue treatment comprising a blood chamber holding whole blood, and a dispensing element configured for dispensing, upon actuating the applicator, a volume of whole blood onto a tissue in need of treatment. In some embodiments, the applicator or chamber comprises an inlet for receiving into said blood chamber a coagulation initiator whereby it is mixed with said whole blood.

In some embodiments, the applicator comprises a first chamber for holding whole blood, a second chamber for holding a coagulation initiator, and a dispensing element configured for concomitantly receiving, upon actuating the applicator, a volume of whole blood from said first chamber and a volume of coagulation initiator from said second chamber and dispensing the two together onto a tissue in need of treatment.

Also disclosed by the present disclosure is a method of tissue treatment comprising dispensing together onto a tissue in need of said treatment a combination of whole blood and a coagulation initiator. The combination is liquid or semi-liquid, as will be further discussed below.

More specifically, the method comprises actuating an applicator as disclosed herein to thereby dispense together, onto the tissue in need of treatment, the whole blood and the coagulation initiator.

In addition, disclosed herein is a kit comprising an applicator for use in tissue treatment as disclosed herein and instructions for use of the applicator for tissue treatment.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 provides a schematic illustration of an applicator in accordance with an embodiment of the present disclosure.

FIGS. 2A-2D are photographic images illustrating different stages of performing the method in accordance with an embodiment of the present disclosure, where a tissue wound on an arm, as shown in FIG. 2A, is sprayed with an applicator 100 shown in FIGS. 2B and 2C that contains whole blood that was mixed with a coagulation initiator prior to spraying, until a thin-layer of the coagulating blood covers the desired area of the issue, as shown in FIG. 2D.

### DETAILED DESCRIPTION OF NON-LIMITING EMBODIMENTS

The present disclosure provides an applicator, method and kit for treating tissue that involves application of liquid or semi liquid whole blood onto the tissue, in combination with a coagulation initiator, such that a layer of clotting/coagulating whole blood is formed, in situ, over the tissue. It has been found that this kind of clotting whole blood supports regeneration of tissue cells, and thus beneficiary in treatment of skin as further discussed below.

In the context of the present disclosure, when referring to “tissue treatment” it is to be understood to encompass any effect of improving the condition of the tissue being treatment, as compared to its condition prior to treatment, the improvement being determined by parameters acceptable with respect to the condition being treatment. The term “treatment” refers to regenerative treatment, in which the tissue being treated is renewed to either restore lost function, to establish normal function, or to enhance a given function.

In a preferred embodiment, the treatment is of human tissue.

In some embodiments, the condition is a wound and the improvement is exhibited by the reduction in severity and/or dimension of the wound. In some embodiments, the wound is a bleeding wound, e.g. as a result of a trauma or following surgery; in some other embodiments, the wound is a non-bleeding wound, e.g. a burn or ulcer.

In some embodiments, the condition is a skin condition, e.g. skin scar, pigmentation, skin scratch, loss of hair, skin wrinkles, acne, suture site, skin donor site (e.g. in the course of skin transplantation), necrotic tissue, etc., each representing a separate embodiment of the present disclosure.

In some embodiments, the condition is sports injury.

In some embodiments, the condition is dental implant.

In some embodiments, the condition is bone implant.

In some embodiments, the condition is involved with junctura problem.

The condition is treated by the combination of whole blood and coagulation initiator, that together cause the formation of a whole blood clot, such that the treatment is manifested by the freshly formed whole blood clot.

The term whole blood refers to (i) blood taken from venous or arterial circulation under sterile conditions and includes all components required for blood coagulation; (ii) blood that has not been modified except for, perhaps, the addition of an anticoagulant or other chemical or biological substances.

The whole blood is typically human blood, withdrawn from a subject under sterile conditions.

In some embodiments, the blood may be fresh blood, i.e. withdrawn from a subject under sterile conditions shortly before use in the treatment. Such blood may or may not include an anti-coagulant to prevent premature coagulation of the withdrawn blood.

In some other embodiments, the blood is one obtained from a blood bank.

In some embodiments, the whole blood is autologous to the subject in need of the tissue treatment.

In some other embodiments, the whole blood is homologous to the subject in need of said tissue treatment.

In some embodiments, the whole blood comprises an anti-coagulant, such as EDTA (ethylenediaminetetraacetic acid), EGTA (ethylene glycol tetraacetic acid), citrate, Heparin oxalate, Batroxobin, vitamin E, Hementin, vitamin K antagonists, Factor Xa inhibitors and Thrombin inhibitors, each representing an independent embodiment of the present disclosure.

As disclosed above, the present disclosure also makes use of one or more coagulation initiators. Blood coagulation initiators are known in the art also by the term clotting agents, and include without being limited thereto, kaolin, Ca²⁺, Mg²⁺, negatively charged phospholipid (PL), protamine sulfate, coagulation factors, Thrombin, vitamin K.

The coagulation initiator may be provided in any form, such as, liquid, fine powder, granulate, etc.

In one embodiment, the blood coagulation initiator comprises at least kaolin. There are many commercially available coagulation initiators based on kaolin, such as, without being limited thereto, QuickClot.

Taking into consideration the above, by a first of its aspects, the present disclosure provides an applicator for use in tissue treatment comprising: a first chamber for holding whole blood, a second chamber for holding one or more coagulation initiator, and a dispensing element configured for concomitantly receiving, upon actuating the applicator, a volume of whole blood from said first chamber and a volume of coagulation initiator from said second chamber and concomitantly dispensing the two together onto a tissue in need of treatment. The dispensing may be directly onto the tissue or into a subsequent device/system from which the coagulating whole blood is then dispensed, as further discussed below.

The applicator is used for the concomitant application of the whole blood and coagulation initiator. When referring to “concomitant” application or dispensing, it is to be understood to refer to the simultaneous delivery (e.g. as a mixture or two discrete volumes), although, according to some embodiment, concomitant may also include the dispensing of one immediately after the other.

In some embodiments, the concomitant delivery is achieved by the operation of an actuator or set of actuators configured for withdrawal of whole blood and coagulation initiator from the respective chambers. The actuator or set of actuators (e.g. one for each chamber) may be of any kind known to be used in liquid-type applicators. In some embodiments, the actuator is of a type used in trigger dispensers, where uses are typically required to grasp the applicator at a neck portion thereof, or head portion thereof and squeeze a trigger. In this mode of operation, small amounts of the contents are dispensed with each squeeze of the trigger.

In some alternative embodiments, the actuator is of a type used in pump dispensers, where the user is required to press down on the actuator.

In some further alternative embodiments, the actuator is of a type used in aerosol sprayers, where the user depresses the actuator.

Unlike trigger and pump dispensers, the contents of aerosol spray applicators are typically continuously dispensed as long as the actuator is depressed or the stem is displaced, such as that used in a trigger dispenser, a pump dispenser.

In some embodiments the actuator is configured for releasing/dispensing discrete volumes of fluid from the applicator. In some other embodiments, the actuator is configured for continuous dispensing of the content (be it the mixture of the blood and coagulation initiator, or only one of the two) as long as the dispensing element is active.

The control of volumes being released can be facilitated by the use of dedicated valves. In some embodiments, each chamber is equipped with a valve e.g. at the connection to the dispensing element, that can be either open or closed to thereby dictate release or no release of content from the respective chamber. In some embodiments, in addition or as an alternative to the chambers' valves, the applicator may be equipped with a volume regulating valve common to both chambers, located between the chambers and the dispensing element, allowing to dictate the amount of fluid dispensed.

Further, in some embodiments, the volume dispensed may be control manually, e.g. by the extent of triggering the actuator or the extent of pressing the chambers per se (the latter being in case of collapsible/compressible chambers).

In some embodiments, the whole blood chamber and/or coagulation initiator chamber each are equipped with an inlet for replenishing the respective chamber with a new amount of content held therein.

In some embodiments, the applicator is one comprising only a blood chamber, and this chamber may include means, e.g. dedicated port, for introducing into the chamber the coagulation initiator where it is mixed with the whole blood in the blood chamber and immediately thereafter dispensed from the applicator as a mixture.

In other embodiments, a ready\premade mixture of blood and coagulation initiator is prepared and essentially immediately is introduced into a chamber for further dispensing the mixture.

In yet some further embodiments, one or both chambers are in the form of disposable or re-fillable cartridges such that once emptied, it can be removed and a new cartridge can be fitted within the applicator. According to this embodiment, the first chamber and/or the second chamber each, independently, are constituted, respectively, by a first cartridge comprising the whole blood and second cartridge comprising the coagulation initiator.

In some embodiments, the applicator comprises additional chamber(s), e.g. for holding a washing liquid for the purpose of washing the applicator's parts, such as tubes, connectors, the dispensing element etc. the washing chamber may be an integral part of the applicator or may be designed to receive a cartridge comprising washing reservoir.

The first and second chambers in the applicator may be of the same or different sizes, and are typically made of or internally coated with a material to which the whole blood and/or coagulation initiator do not adhere. Chambers or containers suitable for holding blood or for holding biocompatible agents, such as coagulation agents, are known and available in the art.

Once the whole blood and/or coagulation initiator is withdrawn from the respective chambers, it may be directly applied onto the tissue via the dispensing element or be introduced into a mixing chamber (an enclosed space where the two materials can be mixed and clotting then starts). Such mixing chamber is in fluid communication with the dispensing element, and the content of the mixing chamber is then immediately/sequentially dispensed via the dispensing element.

In some embodiments, the applicator is equipped with a timer for controlling the time of release of the coagulating whole blood from the mixing chamber into the dispensing element. Such timer may be of importance in order to ensure that the matter being released includes whole blood at the desired coagulation stage, e.g. where the whole blood is sufficiently clotted but not too clotted to cause clogging of the dispensing element. The timer may be mechanical timer or a digital timer and a person versed in the art will be able to dictate the time of dispensing from the mixing chamber, e.g. from 1 second to about 30 seconds, at times from 1 second to 20, or 15 seconds.

The term dispensing element in the context of the present application refers to a mechanical unit configured for concomitantly receiving, upon actuating the applicator, a volume of fluid (typically liquid, although the coagulation may also be dispensed in particulate form), be it the combination/mixture of whole blood and coagulation initiator or separate, and release of same together from the applicator by a mechanism depending on the type of dispensing element used.

In some embodiments, the dispensing element includes a single dispensing head for receiving and dispensing the whole blood and coagulation initiator together. In some other embodiments, the dispensing element includes two or more dispensing heads for the separate receipt, yet concomitant dispensing of the whole blood and coagulation initiator.

In some embodiments, the dispensing element comprises a spray nozzle, e.g. to release the liquids therefrom in a form of an aerosol.

In some other embodiments, the dispensing element comprises a brush.

In yet some other embodiments, the dispensing element comprises a dripping tip.

In some embodiments, the dispensing element is configured for being mounted onto capital equipment suitable for dispensing the coagulating whole blood. As such, the applicator may be a disposable unit that is used as a cartridge for multi-use and sophisticated dispensing systems. Examples of a multi-use systems that can be used together with the applicator disclosed herein include airbrush units, timers (to be coupled to the/a dispensing element), volume control units (for controlling the volume of the coagulation initiator and/or whole blood discharged from the two chambers), pressure control units (for controlling the pressure at which the coagulation initiator and/or whole blood are discharged from the two chambers), etc.

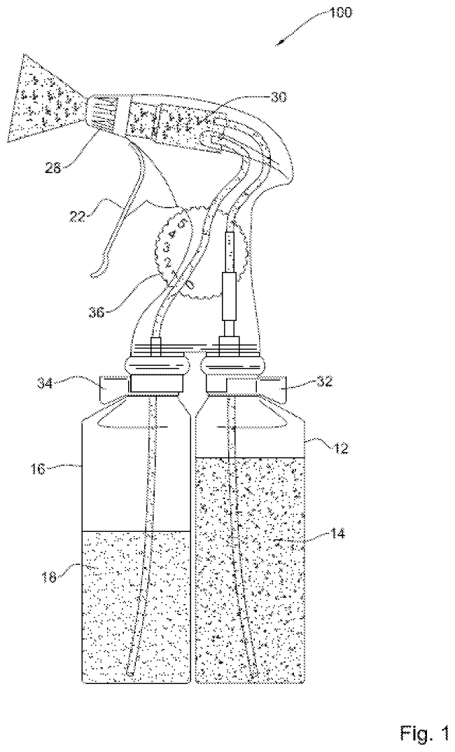

For demonstrating the structural elements of an applicator according to one embodiment disclosed herein, reference is made to FIG. 1. It is to be noted that this Figure is intended for illustration of an exemplary embodiment of the broader context of the present disclosure and is not intended to be limiting.

Specifically shown in FIG. 1 is an applicator 10, comprising a first chamber 12 holding whole blood 14, a second chamber 16 holding a coagulation initiator 18, and a dispensing element 20 configured for concomitantly receiving, upon actuating the applicator through actuator 22, a volume of whole blood from the first chamber 12, being delivered through a first delivery tube 24 and a volume of coagulation initiator from the second chamber 16, being delivered through a second delivery tube 26 and dispensing the two together via a spray nozzle 28, onto a tissue in need of treatment.

Applicator 10 also includes a mixing chamber 30, for mixing the volume of whole blood and volume of coagulation initiator prior to dispensing the same through spray nozzle 28.

In addition, Applicator 10 includes, respectively, first and second valves 32 and 34, for closing or opening, independently, each of the respective first and second chambers. For example, it may be that there is a need to dispense only one of the blood or coagulation initiator, and accordingly, the valve may be open or close, according to need. This may be the case, where, for example, after a first dispense of the mixture of whole blood and coagulation initiator to form a first layer of clotting/coagulating blood onto the tissue, a second layer of only blood or only coagulation initiator is needed.

Applicator 10, according to this embodiment, also includes a volume regulator 36 for controlling the volume of whole blood and/or coagulation initiator being withdrawn from their respective chambers 12 and 16.

The present disclosure also provides, by a second of its aspects, a method of tissue treatment comprising dispensing together onto the tissue whole blood and a coagulation initiator. In some embodiments, the whole blood and the coagulation initiator are mixed at a location remote from the tissue, e.g. in an applicator, as disclosed herein. In some other embodiments, the whole blood and the coagulation initiator are applied onto the tissue in sequence, i.e. one very shortly after the other.

When using the applicator disclosed herein for performing the method of tissue treatment, this is typically achieved by actuating the actuator which causes the whole blood preferably together with the coagulation initiator to be dispersed over the treated tissue. The operation of the applicator is evident from the type of applicator used as above disclosure.

For example, if the applicator comprises a mechanism for releasing content from the chambers by pressing the chambers and the dispensing element comprises a brush, the treatment may involve brushing the mixture of whole blood and coagulation initiator expelled from the applicator, over the tissue.

Further, for example, if the applicator comprises a spraying mechanism for releasing content from the chambers, the treatment may involve spraying small droplets or aerosol of the mixture over the tissue.

The application of the whole blood and coagulation initiator causes clotting of the whole blood. While the clotting cascade may begin within the applicator, the whole blood is dispensed in a liquid or semi-liquid form such that a layer of clotting/coagulating whole blood is formed over the tissue. Thus, in the context of the present disclosure, when referring to clotting whole blood it is to be understood as whole blood in which the clotting cascade has begun, but the whole blood is still in a physical state that it can be sprayed or dripped or brushed etc., over the tissue.

In some embodiments, the method comprises applying two or more layers of clotting whole blood over the treated tissue. The layers of clotting whole blood may then be covered by a wound dressing, such as a gauze, bandage, or plaster.

As noted above, the whole blood may be autologous or homologous to the subject receiving the tissue treatment. Accordingly, the method may comprise also a step of sterile withdrawal of blood from a subject, be it a donor subject of the subject undergoing treatment. The withdrawn blood can then be immediately introduced into the applicator for use in the tissue treatment, or be mixed with an anti-coagulant, to permit later use of the withdrawn whole blood.

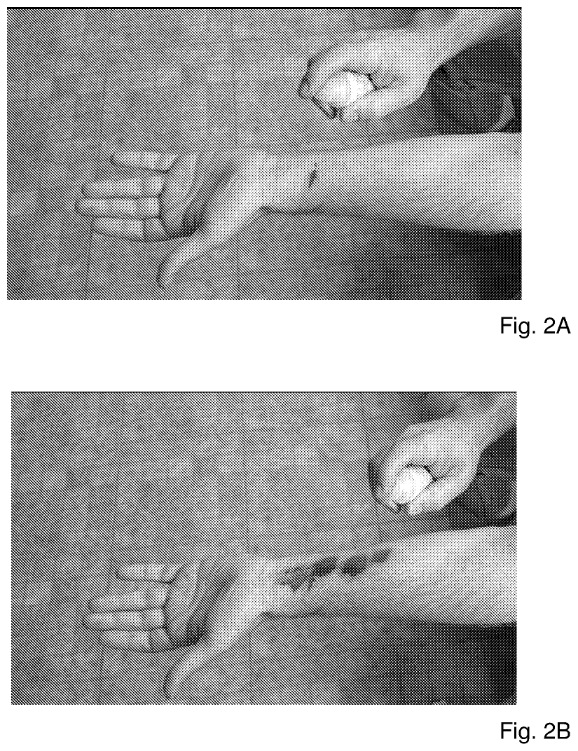

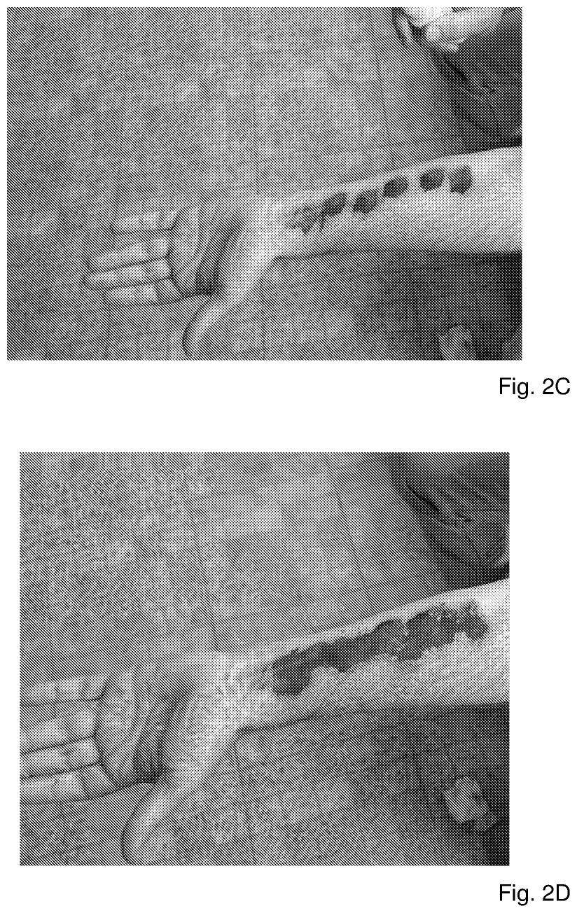

Reference is now made to FIGS. 2A-2D showing images of the method disclosed herein, according to one embodiment. Specifically shown is the use of a sprayer dispenser (pump-type dispenser) containing a mixture of whole blood and kaolin (not shown) and the pressing of an actuator to cause discrete amounts of the mixture to be applied onto the skin, as shown by the circular blood spots on the skin.

FIG. 2A shows a bleeding skin wound prior to treatment. Then a series of discrete dispensing events were used to obtain continuous coverage of a desired skin area, as shown in the sequence of images provided in FIGS. 2B-2D.

The method may be performed using a dedicated kit, which is also provided by the present disclosure. Specifically, disclosed herein is a kit comprising the applicator as disclosed herein.

In some embodiments, the kit comprises instructions for use of the applicator for tissue treatment.

In some additional embodiments, the kit comprises blood withdrawal elements for sterile withdrawal of whole blood from a subject in need of the tissue treatment and for introducing the same into the first chamber.

In some embodiments, the kit comprises one or more cartridges holding the coagulation initiator.

1. An applicator for use in tissue treatment comprising:
a first chamber for holding whole blood,
a second chamber for holding a coagulation initiator, and
a dispensing element configured for concomitantly receiving, upon actuating the applicator, a volume of whole blood from said first chamber and a volume of coagulation initiator from said second chamber and dispensing the two together onto a tissue in need of treatment.

2.-3. (canceled)

4. The applicator of claim 1, comprising an actuator for actuating said applicator according to need or the applicator is actuated by concomitantly depressing the first chamber and the second chamber, and wherein said dispensing element comprises a mixing chamber for receiving and mixing said volume of whole blood and said volume of coagulation initiator before dispensing the same.

5. The applicator of claim 1, wherein said dispensing element comprises a spray nozzle.

6. The applicator of claim 1, wherein said dispensing element comprises a brush.

7. The applicator of claim 1, wherein said dispensing element comprises a dripping tip.

8. The applicator of claim 1, wherein said first chamber is constituted by a first cartridge comprising the whole blood and/or wherein said second chamber is constituted by a second cartridge comprising the coagulation initiator.

9.-11. (canceled)

12. The applicator of claim 8, wherein said whole blood is autologous to the subject in need of said tissue treatment, or homologous to the subject in need of said tissue treatment.

13. (canceled)

14. A method of tissue treatment comprising dispensing together onto the wound whole blood and a coagulation initiator, the method comprising
actuating an applicator comprising:
a first chamber for holding whole blood,
a second chamber for holding a coagulation initiator, and
a dispensing element configured for concomitantly receiving, upon actuating the applicator, a volume of whole blood from the first chamber and a volume of coagulation initiator from the second chamber and dispensing the two together;
wherein the whole blood and the coagulation initiator are dispensed together onto the tissue in need of treatment.

15. (canceled)

16. The method of claim 14, comprising actuating an applicator comprising a blood chamber holding whole blood, and a dispensing element configured for dispensing, upon actuating the applicator, a volume of whole blood from the blood chamber mixed with a volume of coagulation initiator.

17. The method of claim 14, comprising actuating the applicator by pressing an actuator of the applicator.

18. The method of claim 14, comprising actuating the applicator by concomitantly depressing the first chamber and the second chamber.

19. The method of claim 14, wherein the applicator comprises a mixing chamber for receiving and mixing said volume of whole blood and said volume of coagulation initiator before dispensing the same.

20. The method of claim 14, wherein the dispensing is by spraying through a spray nozzle.

21. The method of claim 14, wherein the dispensing element comprises a brush and the dispensing is by a brushing together the whole blood and coagulation initiator over the tissue.

22. The method of claim 14, wherein the dispensing element comprises a dripping tip and the dispensing is by dripping together the whole blood and coagulation initiator over the tissue.

23. The method of claim 14, wherein the first chamber constitutes a first cartridge comprising whole blood and said method comprises replenishing first cartridge according to need.

24. The method of claim 14, wherein the second chamber is constituted by a second cartridge comprising the coagulation initiator and said method comprises replenishing the second cartridge according to need.

25. The method of claim 14, comprising introducing into the first chamber whole blood withdrawn from a subject under sterile conditions.

26.-28. (canceled)

29. A kit comprising:
(i) an applicator for use in tissue treatment comprising:
a first chamber for holding whole blood,
a second chamber for holding a coagulation initiator, and
a dispensing element configured for concomitantly receiving, upon actuating the applicator, a volume of whole blood from said first chamber and a volume of coagulation initiator from said second chamber and dispensing the two together;
(ii) instructions for use of the applicator for tissue treatment.

30. The kit of claim 29, comprising blood withdrawal elements for sterile withdrawal of whole blood from a subject in need of the tissue treatment and for introducing the same into the first chamber.

31. A kit comprising:
(i) an applicator for use in tissue treatment comprising:
a blood chamber holding whole blood,
a container holding a coagulation initiator, and
a dispensing element configured for receiving, upon actuating the applicator, a volume of whole blood mixed with a volume of coagulation initiator and dispensing the two together;
(ii) instructions for use of the applicator for tissue treatment, the instructions comprise mixing a volume of coagulation initiator into said blood chamber prior to actuating the applicator.
